(19) United States
(12) Patent Application Publication
(10) Pub. No.: US 2003/0211536 A1
(43) Pub. Date: Nov. 13, 2003

(54) Systems and methods to facilitate multiple order combinatorial chemical processes

(76) Inventor: H. Mario Geysen, Charlottesville, VA (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/148,057

(22) PCT Filed: Dec. 5, 2000
(86) PCT No.: PCT/US00/32936

(51) Int. Cl. G01N 33/53; G01N 33/543

(52) U.S. Cl. 435/7.1; 436/518

(57) ABSTRACT

A method to screen for reactive chemicals comprises the steps of configuring a set of constructs such that each construct of the set includes a pairwise combination of a chemical entity (A₁-Aᵢ) of a chemical library (A) and a chemical entity (B₁-Bⱼ) of a chemical library (B). The set of constructs includes essentially every possible pairwise combination of the chemical entities (A₁-Aᵢ) of the chemical library (A) and the chemical entities (B₁-Bⱼ) of the chemical library (B). The constructs are exposed to a given set of conditions to facilitate reactions or interactions between the chemical entity (A₁-Aᵢ) and the chemical entity (B₁-Bⱼ) of each construct. The constructs are screened to identify any reactions or interactions, and the chemical entity (A₁-Aᵢ) and the chemical entity (B₁-Bⱼ) of any constructs where reactions or interactions occurred are identified.

### BACKGROUND OF THE INVENTION

This invention relates generally to the field of chemistry, and in particular to techniques for synthesizing chemical entities and evaluating reactions between the chemical entities when subjected to certain reaction conditions. More specifically, the invention relates to the creation of diverse chemical libraries and to the identification of reactions that occur between members of the libraries.

Recent trends in the area of research for novel chemicals, including pharmacological agents, have been concentrated on the preparation of so-called “chemical libraries”. Chemical libraries are intentionally created collections of differing molecules or chemical entities which can be prepared either synthetically or biosynthetically. Following synthesis, the chemical entities may be used in various assays or combined with other chemicals and then screened for biological activity or chemical reactivity.

One way to produce chemical libraries is by synthesizing the various chemicals to individual solid supports, which typically take the form of resin beads. A variety of techniques have been proposed for making chemical libraries which utilize individual solid supports to which the chemicals are tethered. One such method is the “discrete” method where solid supports are placed into multiple reaction vessels. Various chemicals are then synthesized onto the solid supports while the solid supports remain within the reaction vessels. After completing the synthesis process, the chemical compound on each solid support may be identified simply by identifying the reaction vessel from which the solid support was removed. Because of the need to maintain the solid supports within a given reaction vessel, the size of the resulting chemical library is limited by the number of reaction vessels used.

In an attempt to greatly increase the size of a chemical library, the mix and split technique was developed. In the mix and split method, solid supports are placed into individual reaction vessels and a first building block is synthesized onto each of the solid supports. Solid supports are then mixed together and redistributed to the reaction vessels where a second building block is synthesized onto the solid supports. The solid supports may once again be mixed and redistributed where another building block may be synthesized onto the solid supports. This process may be repeated as many times as necessary. Examples of mix and split techniques are described generally in U.S. Pat. No. 5,503,805, the complete disclosure of which is herein incorporated by reference.

Once a mix and split chemical library has been produced, the compound may be cleaved from the solid supports and tested to determine if the compound produces a desired result. If so, the particular compound needs to be identified. However, since the solid support was mixed and split one or more times during the synthesis process, identifying the compound on the solid support can be challenging. A variety of techniques have been proposed for identifying the compounds, such as by the use of identifier tags as described in U.S. Pat. No. 5,708,153, or by the use of identification codes as described generally in PCT International Application No. PCT/US97/05701, and in H. Mario Geysen, et al., Isotope or Mass Encoding of Combinatorial Libraries, Chem. & Biol. Vol. III, No. 8, pp. 679-688, August 1996, the complete disclosures of which are herein incorporated by reference.

Although a variety of techniques exist for creating diverse chemical libraries and for identifying the resulting chemical entities of the libraries, little has been done in the way of improving the efficiency of processes that utilize the resulting chemical libraries. For example, it may be desirable to attempt to react the chemical entities of one library with the chemical entities of another library. For instance, it may be desirable to attempt to react multiple catalysts with a chemical library to evaluate the usefulness of various catalysts.

To perform such reactions using existing techniques, the chemicals are typically cleaved from their solid supports and then combined in a well with cleaved chemicals from another library under certain reaction conditions. If reactivity occurs, the combined chemicals still need to be identified as previously mentioned. Unfortunately, such a process can be unpractical for even moderately sized libraries. For example, if each library had 1,000 members, then the total number of required reactions would be 1,000,000. Individual cleavage and placement of chemicals into 1,000,000 wells is simply impractical.

Hence, the invention is related to techniques and chemical constructs which enable multiple chemical libraries to be created, reacted with each other and screened in an efficient manner. Once chemical reactivity has been identified, the invention also provides techniques for identifying the particular chemical entities involved in the reactions.

### SUMMARY OF THE INVENTION

The invention provides various screening techniques along with novel constructs that may be used when screening for reactive chemicals. In one specific embodiment, a method is provided to screen for reactive chemicals by configuring a plurality of constructs such that each construct of the set includes a pairwise combination of a chemical entity A₁-Aᵢ of a chemical library A and a chemical entity B₁-Bⱼ of a chemical library B. Further, the set of constructs include essentially every possible pairwise combination of the chemical entities A₁-Aᵢ of the chemical library A and the chemical entities B₁-Bⱼ of the chemical library B. In this way, each chemical entity from library A and from library B are unambiguously associated, e.g. on a solid support, so that every possible combination of chemical entities from two or more libraries may be tested for reactivity. The constructs are exposed to a given set of conditions to facilitate a reaction or interaction between the chemical entity A₁-Aᵢ and the chemical entity B₁-Bⱼ of each construct. The constructs are then screened to identify where a reaction or an interaction occurred. If any reactions or interactions are identified, the chemical entity A₁-Aᵢ and the chemical entity B₁-Bⱼ of the associated constructs are determined.

In one aspect, each construct includes at least a pair of sites. Further, the chemical entity A₁-Aᵢ is synthesized to one of the sites of each construct while the other site is blocked. The other site of each construct is then unblocked, and the chemical entity B₁-Bⱼ is synthesized to the other site of each construct. In another aspect, the constructs are formed using a combinatorial processes to achieve the desired pairwise combinations. For example, the constructs may be mixed together after synthesizing the chemical entities A₁-Aᵢ and then split into groups such that each group has constructs with essentially all other chemical entities A₁-Aᵢ. The chemical entities B₁-Bⱼ may then be synthesized onto the constructs such that each group receives a different chemical entity B₁-Bⱼ. Optionally, further combinatorial processes may be used when synthesizing library A and/or library B onto the constructs. For example, a combination of chemicals may be synthesized on each construct to create each A₁-Aᵢ chemical entity and each B₁-Bⱼ chemical entity, e.g. using a mix and split technique. In this way, the chemical entities of each library may be constructed of a single chemical building block or multiple chemical building blocks.

In another aspect, the constructs are screened by sensing for a change in temperature to indicate that a reaction or an interaction has occurred. Screening may also be accomplished by measuring for the mass of any chemical products, e.g. using a mass spectrometer. Other screening techniques include the use of ultraviolet light to test for a color change or phosphorescence resulting from the creation of a chemical product, colored chromophotography, and the like.

In still another aspect, the specific chemical entities may be determined by evaluating the masses of the unreacted chemical entities A₁-Aᵢ and the unreacted chemical entities B₁-Bⱼ using mass spectrometry, and correlating each mass with an associated chemical entity of each library, e.g. by using a look-up table. Alternatively, each chemical entity A₁-Aᵢ and each chemical entity B₁-Bⱼ may be encoded with a code. The codes may then be decoded to determine the specific chemical entities. Conveniently, the codes may be decoded by evaluating the mass of the codes using mass spectrometry and correlating each mass with an associated chemical entity, e.g. by using look-up tables.

In one particular aspect, the chemical entities A₁-Aᵢ or B₁-Bⱼ comprise catalysts. In this way, multiple chemicals may be reacted with multiple catalysts in an efficient manner. In another particular aspect, multiple libraries of constructs are provided that each include the same pairwise combinations of chemical entities A₁-Aᵢ chemical entities B₁-Bⱼ. Further, each library of constructs is exposed to a different set of conditions. In this way, multiple libraries of chemicals may be exposed to multiple conditions in a high throughput manner. In an alternative aspect, each library of constructs may be exposed to a metal in one of its oxidation states as part of a third or higher order combinatorial process.

The invention further provides a method for making a library of constructs. The constructs may be formed on a plurality of solid supports that each include at least two sites. A chemical entity from a chemical library A having A₁-Aᵢ chemical entities is synthesized to one of the sites of each solid support while the other site is blocked. The blocked site for each solid support is then unblocked and a chemical entity from a chemical library B having B₁-Bⱼ chemical entities is synthesized to the unblocked sites. The chemical entities A₁-Aᵢ and the chemical entities B₁-Bⱼ are synthesized to the sites to form a set of constructs that includes essentially every possible pairwise combination of the chemical entities A₁-Aᵢ of the chemical library A and the chemical entities B₁-Bⱼ of the chemical library B.

In one aspect, the constructs are mixed after synthesizing the chemical entities A₁-Aᵢ and are split into groups such that each group has constructs with essentially all other chemical entities A₁-Aᵢ. The chemical entities B₁-Bⱼ are then synthesized onto the constructs such that each group receives a different chemical entity B₁-Bⱼ. Optionally, the chemicals may be synthesized by synthesizing a combination of chemicals on each construct to create each A₁-Aᵢ chemical entity and each B₁-Bⱼ chemical entity. In this way, each chemical entity may be constructed of a single chemical building block or multiple building blocks. Conveniently, a mix and split technique may be employed to synthesize the chemicals. Optionally, each chemical entity A₁-Aᵢ and each chemical entity B₁-Bⱼ may be encoded with an identification code to facilitate identification of the chemical entities following screening.

The invention further provides an exemplary construct that comprises a solid support having at least one arm and two or more sites branching from the arm. A chemical entity A is coupled to one of the sites, and a chemical entity B is coupled to the other site. Further, the pair of sites are configured such that the chemical entity A is spaced apart from the chemical entity B at a distance selected to facilitate a reaction between the chemical entity A and the chemical entity B. Optionally, an identification code may be coupled to the chemical entity A and the chemical entity B.

In another embodiment, the invention provides a library of chemical constructs. The library includes a set of constructs that each comprise a solid support having at least one arm and at least a pair of sites branching from the arm. A chemical entity A₁-Aᵢ of a chemical library A is coupled to one of the sites, and a chemical entity B₁-Bⱼ of a chemical library B is coupled to the other site. Further, the pair of sites are configured such that each chemical entity A₁-Aᵢ is spaced apart from each chemical entity B₁-Bⱼ at a distance selected to facilitate a reaction or an interaction between each chemical entity A₁-Aᵢ and each chemical entity B₁-Bⱼ.

In one aspect, the set of constructs includes essentially every possible pairwise combination of the chemical entities A₁-Aᵢ of the chemical library A and the chemical entities B₁-Bⱼ of the chemical library B. In another aspect, the chemical entities A₁-Aᵢ of the chemical library A and/or the chemical entities B₁-Bⱼ of the chemical library B each comprise multiple chemical building blocks that have been synthesized to the sites. In still another aspect, the library A or the library B comprises catalysts.

In an alternative embodiment, a library of chemical constructs comprises a set of constructs that each comprise a solid support having at least a pair of sites. A chemical entity A₁-Aᵢ of a chemical library A is coupled to one of the sites, with the chemical entity A₁-Aᵢ comprising two or more chemical building blocks that have been synthesized to the site. A chemical entity B₁-Bⱼ of a chemical library B coupled to the other site. For example, the chemical library B may comprise catalysts. Optionally, the chemical entity B₁-Bⱼ may also be constructed of two or more building blocks.

In one aspect, the set of constructs includes essentially every possible pairwise combination of the chemical entities A₁-Aᵢ of the chemical library A and the chemical entities B₁-Bⱼ of the chemical library B. In another aspect, an identification code may be coupled to each of the chemical entities.

The invention further provides techniques for evaluating combinations of catalysts to determine which combinations are the most efficient in producing end products. For example, in one method, a set of constructs are configured such that each construct of the set includes a pairwise combination of a chemical entity A₁-Aᵢ of a catalyst library A and a chemical entity B₁-Bⱼ of a catalyst library B. The constructs are exposed to a substrate in solution phase to facilitate potential reactions involving the chemical entity A₁-Aᵢ and the chemical entity B₁-Bⱼ of each construct. The constructs may then be screened to identify any reactions or interactions, and the chemical entity A₁-Aᵢ and the chemical entity B₁-Bⱼ of any constructs where reactions or interactions occurred may be identified. In one aspect, the set of constructs may include essentially every possible pairwise combination of the chemical entities A₁-Aᵢ of the catalyst library A and the chemical entities B₁-Bⱼ of the catalyst library B. In this way, a comprehensive analysis of the reaction or interaction of two catalysts libraries with a substrate may be performed.

### BRIEF DESCRIPTION OF THE DRAWINGS

FIG. 1 is a schematic view of a solid support having a plurality of tether sites according to the invention.

FIG. 2 is a graph illustrating the distribution of distances between tether sites for the solid support of FIG. 1.

FIG. 3 is a schematic view of a solid support having an alternative arrangement of tether sites according to the invention.

FIG. 4 is a graph illustrating the distance distribution between chemical entities for the solid support of FIG. 3.

FIG. 5A is a flow chart illustrating a second order reaction process according to the invention.

FIG. 5B is a flow chart illustrating a second order catalysis process according to the invention.

FIG. 6 is a flow chart illustrating a third order combinatorial process according to the invention.

FIG. 7 is a schematic view of an analytical construct according to the invention.

FIG. 7A illustrates a process for making one specific analytical construct according to the invention.

FIG. 7B illustrates one specific example of an alternative analytical construct according to the invention.

FIG. 8 is a flow chart illustrating one possible method for synthesizing a chemical library A onto solid supports according to the invention.

FIG. 9 is a schematic view of a library of constructs created from two chemical libraries, A and B.

FIG. 10 illustrates a method for producing the library of constructs of FIG. 9.

FIG. 11 illustrates a method where the chemical entities on a library of constructs are tested for reactions or interactions by subjecting the constructs to various conditions.

FIG. 12 illustrates a resulting product C when two chemical entities A and B react or interact.

FIG. 13 illustrates a method for producing a library of constructs and identifying reacting or interacting chemicals.

FIG. 13A illustrates a screening method to screen for reactive or interactive chemicals.

FIG. 14 is a graph produced when analyzing one of the constructs of FIG. 10 after a reaction or interaction has occurred using mass spectroscopy.

FIG. 15 illustrates a look-up table associating atomic mass units with chemical entities.

FIG. 16 is a schematic view of an alternative construct having codes associated with the chemical entities according to the invention.

FIG. 17 illustrates a method for producing a mass encoded library of constructs and using mass codes to identify reacting or interacting chemicals.

FIG. 18 is a graph produced when analyzing one of the constructs of FIG. 16 after a reaction or interaction has occurred using mass spectroscopy.

FIG. 19 illustrates a look-up table associating atomic mass units with codes.

FIG. 20 illustrates a method for producing a chemical library involving coordination complexes having metals placed in their centers.

FIG. 21 illustrates a method for producing and evaluating constructs having catalysts when using mass codes.

FIG. 22 illustrates a method for producing a chemical library and then reacting or interacting the chemicals on the constructs using a variety of conditions.

FIG. 23 is a schematic view of an analytical construct having chemical entities from two separate libraries and a reagent.

FIG. 24 illustrates a method for producing and evaluating a chemical library involving multiple catalysts.

### DESCRIPTION OF THE SPECIFIC EMBODIMENTS

The invention provides for the creation of libraries of chemical constructs using chemical entities (i.e., single chemical building blocks or two or more synthesized chemical building blocks) from two or more chemical libraries in order to determine reactivity or interactivity between the chemical entities of one library with the chemical entities of another library. For example, in one embodiment, the invention provides a set of constructs that each have a member of a chemical library A and a member of a chemical library B. The set of constructs are configured such that each member of library A is unambiguously associated with one of the members of library B. In this way, reactions may be attempted between each member of library A with each member of library B.

The constructs of the invention may comprise any chemical arrangement that allows for the attachment of one or more chemical entities. For example, the constructs may comprise solid supports which include one or more tether sites. The constructs may further comprise chemical entities that are linked or tethered to the various sites. The solid supports of the invention may be constructed from one or more materials upon which combinatorial chemistry synthesis can be performed. Example of solids supports that may be used include beads, solid surfaces, solid substrates, particles, pellets, discs, capillaries, hollow fibers, needles, solid fibers, cellulose beads, pore glass beads, silica gels, polystyrene beads optionally crosslinked with divinylbenzene, grafted copoly beads, polyacrylamide beads, latex beads, dimethylacrylamide beads, optionally cross-linked N,N′-bis-acryloyl ethylene diamine, glass particles coated with a hydrophobic polymer, fullerenes and soluble supports, such as low molecular weight, noncrosslinked polystyrene, and the like.

One convenient way for producing the constructs is by linking the chemical entities from library A to one or more sites of the solid supports while other sites are blocked, e.g. with a protecting group. The remaining sites are unblocked and the chemical entities from library B are linked to the remaining sites. The members of the libraries may synthesized to the sites in relatively close proximity to facilitate reactions between the members of the different libraries.

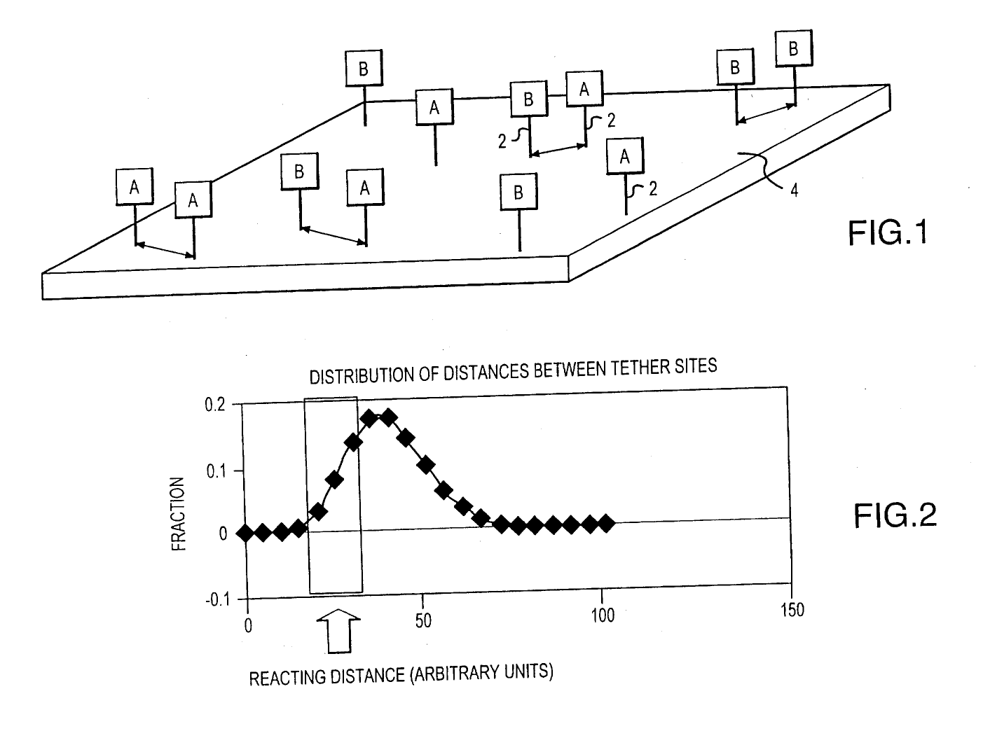

One way of arranging sites 2 on a solid support 4 to facilitate reactions is shown in FIG. 1. Sites 2 are randomly assigned, and a sufficient number of sites are provided so that a reasonable number of the members of libraries A and B are within reacting distance as shown in the graph of FIG. 2. In this way, if the members on a given solid support are reactive, enough of the members will react to permit adequate screening and identification. As shown in the example of FIG. 2 (which includes arbitrary units), the reacting distance may be in the range from about 15 distance units to about 30 distance units. FIG. 2 also illustrates the fraction of sites 2 that fall within the range.

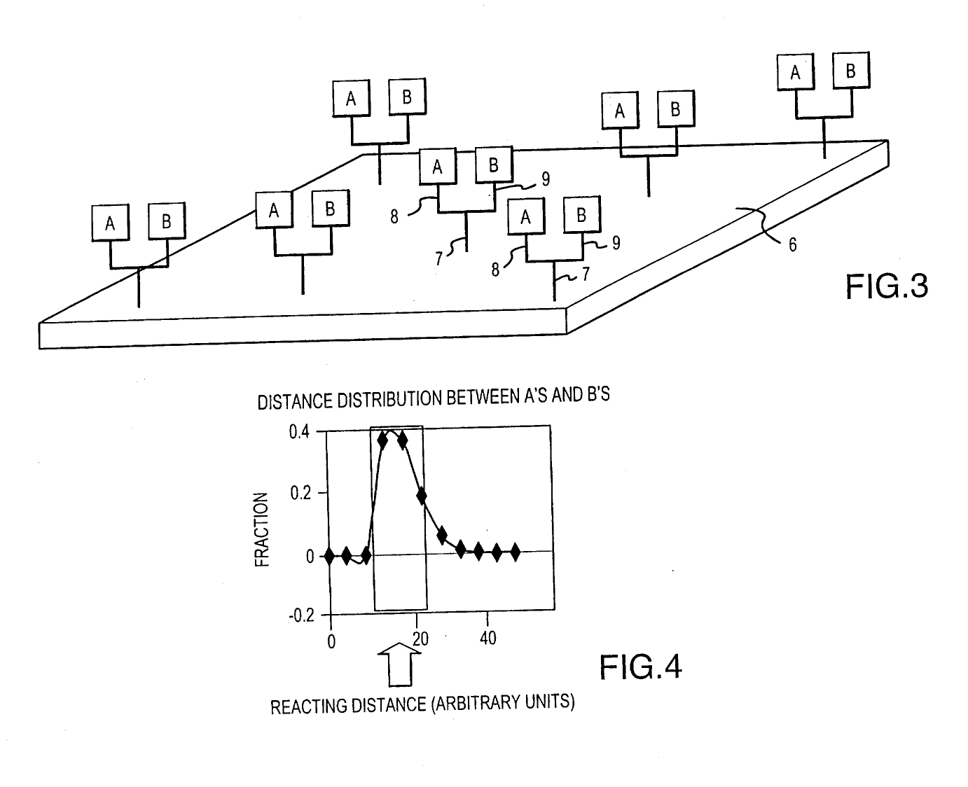

FIG. 3 illustrates another arrangement of tether sites on a solid support 6 to facilitate reactions between the members of the different libraries. Solid support 6 includes multiple arms 7 that each have a pair of sites 8 and 9 branching from arms 7. Sites 8 and 9 may be constructed to maximize the potential for reactions or interactions occurring between the chemical entities. For example, the sites may be constructed to maximize the time averaged fraction of chemical entities that will be within the reacting or interacting range as shown in FIG. 4. The manner of constructing the sites so as to optimize the time averaged fraction is described in greater detail hereinafter.

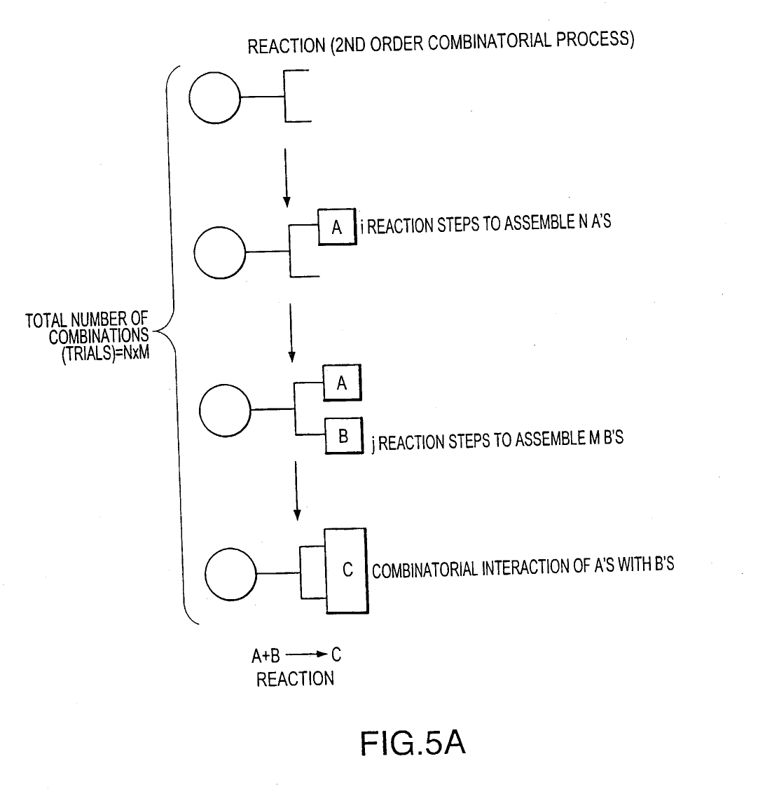

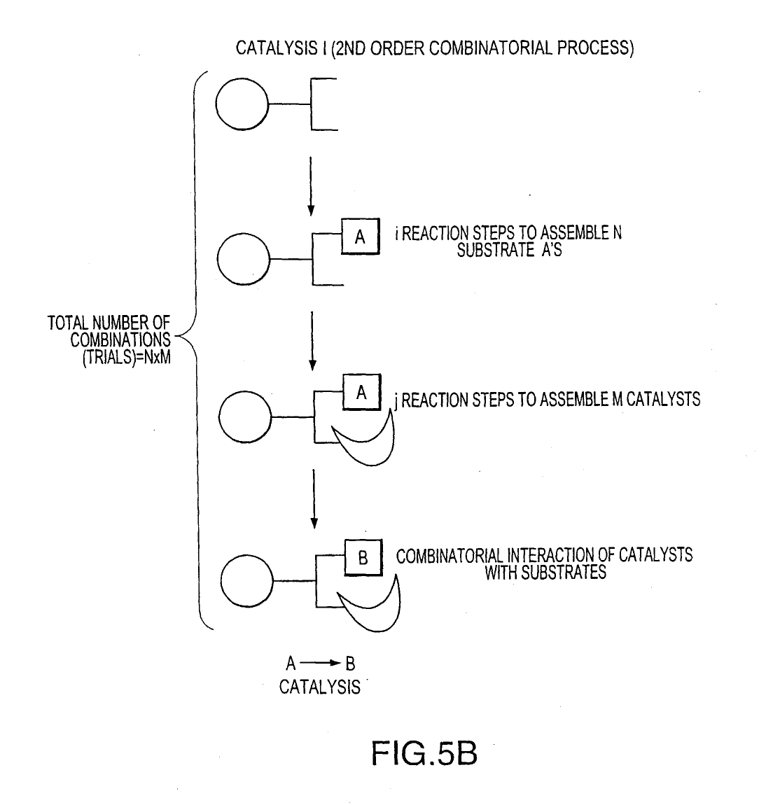

The members of each chemical library may comprise a single chemical building block or may be a synthesized chemical entity formed from two or more monomers or chemical building blocks. Conveniently, mix and split techniques may be employed when synthesizing multiple building blocks onto the tether sites of the solid supports. In this way, two or more relatively large chemical libraries may be reacted with each other in a rapid and convenient manner. For example, as shown in FIG. 5A, a chemical library A may be formed such that each member A₁₋ᵢ is constructed from three building blocks (X,Y,Z). If each building block comprises ten chemicals, then three mix and split steps will result in chemical library A having 10³ members. If a similar process were followed for a chemical library B, it would also have 10³ members. By associating every member of library A with every member of library B, 10⁶ different combinations are provided. Hence, 10⁶ potential reactions or interactions may be evaluated. As shown in FIG. 5B, a similar process may be used with a chemical library A and a catalysis library.

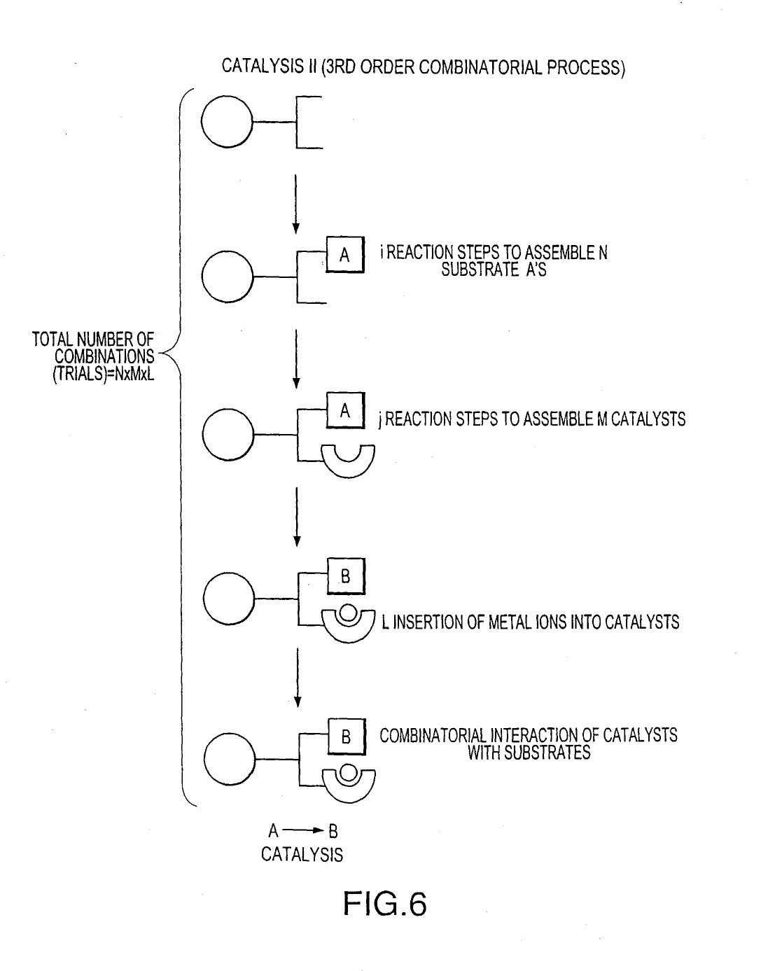

Third or higher order combinatorial processes are also possible. For example, as shown in FIG. 6, a set of constructs that includes a library of catalysts may be subjected to another combinatorial process where the constructs may be loaded/reacted with metals in one or more of their possible oxidation states in order to insert metal ions into the catalysts. Hence, using the example of FIG. 5, if 10³ metals were used, then the number of potential reactions becomes 10⁹.

After forming the constructs, the chemical entities from each of the libraries may subjected to certain conditions to determine if any of the chemical entities are reactive or interactive with each other. Hence, reactions between two or more different chemical libraries may be attempted simply by synthesizing the chemical entities from each of the libraries onto constructs such that each chemical entity from each library is associated with all other entities from all other libraries The constructs are then subjected to appropriate conditions to provide an environment where the chemical entities on the constructs may potentially react or interact with each other.

The constructs may be screened to determine the constructs where chemical reactions or interactions occurred. A variety of techniques may be employed to screen for chemical reactivity or interactivity, including the use of thermography as described generally in Steven J. Taylor, et al., “Thermographic Selection of Effective Catalysts from an Encoded Polymer-Bound Library”, Science, Volume 280, pp. 267-270, Apr. 10, 1998, the complete disclosure of which is herein incorporated by reference. Another screening technique is to clip the link with the solid support and use mass spectroscopy to evaluate whether any chemical products were produced. Use of mass spectrometry as a screening tool may also be used to identify any starting materials as described below. Another screening technique is the use of ultraviolet light to test for a color change or phosphorescence of any products. Such a process may be rapidly accomplished using, for example, a FACS sorter. Still another screening technique utilizes colored chromophotography as described generally in Matthew T. Burger, et al., “Enzymatic, Polymer-Supported Formation of an Analog of the Trypsin Inhibitor A90720A: A Screening Strategy for Macrocyclic Peptidase Inhibitors”, J. Am. Chem. Soc. 1997, 119, 12697-12698.

The constructs experiencing chemical reactions or interactions may be separated out for analysis to identify the chemical entities that were reactive. A variety of techniques may be employed, alone or in combination, to identify the chemical entities. Such techniques include, for example, the measurement of any unreacted chemicals using mass spectroscopy, the use of mass based codes, performing one or more synthesizing steps as discrete steps, and the like.

The invention may be employed to attempt to react or interact a variety of chemical libraries. The chemical libraries included on the constructs may be those creating using any type of synthesis, including combinatorial synthesis processes, as known in the art. As one specific example, one of the libraries may comprise a group of catalysts that are reacted with a group of chemical entities. As another example, the constructs may also include various reagents that may be involved in the reactions. Hence, with the techniques of the invention, multiple combinatorial chemical libraries may be reacted or interacted, screened and evaluated in a rapid and efficient manner.

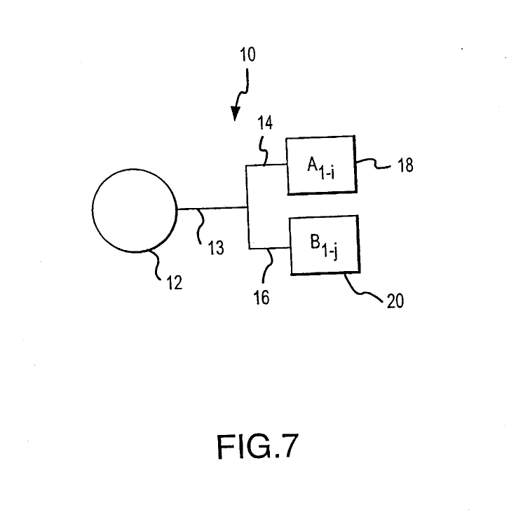

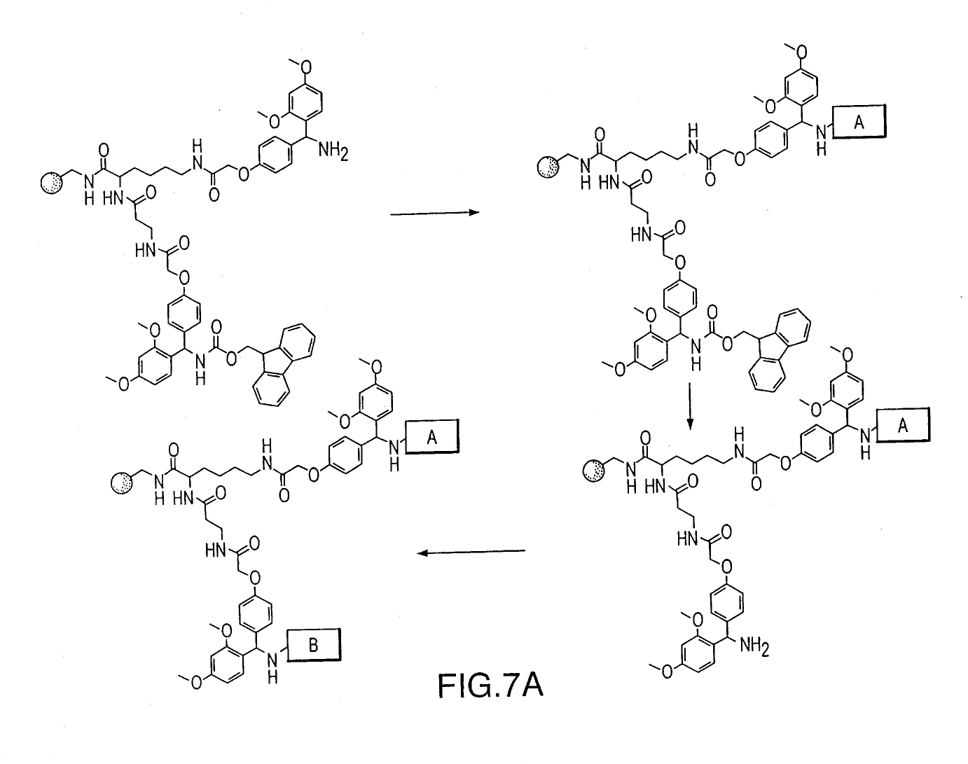

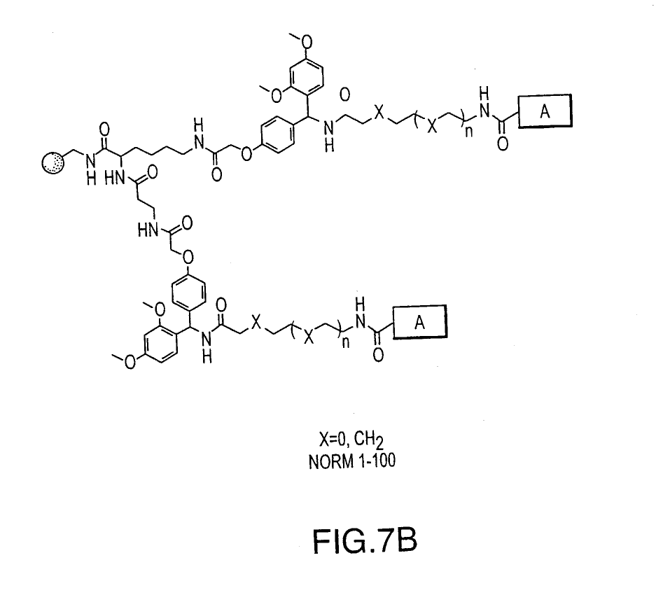

Referring now to FIG. 7, one embodiment of an analytical construct 10 will be described. Construct 10 comprises a solid support 12 that has been engineered to include an arm 13 that has two branching tether sites 14 and 16. Although only one arm is shown, it will be appreciated that multiple arms and associated tether sites may be provided on solid support 12.

Coupled to site 14 is a chemical entity 18, and coupled to site 16 is a chemical entity 20. Chemical entity 18 may comprise any chemical entity from a library A of chemical entities A₁-Aᵢ, and chemical entity 20 may comprises an chemical entity from a library B of chemical entities B₁-Bⱼ (where i and j may or may not be equal).

Sites 14 and 16 are configured to maximize the probability that chemical entities 18 and 20 will be within reacting distance for a sufficient time to permit reactions or interactions to occur, i.e., sites 18 and 20 may be configured to optimize the time averaged distance between the sites to increase the probability that an observable reaction or interaction will occur. Under appropriate conditions, at least some of the chemical entities will react or interact with each other to form a product, or, if one of the libraries comprises catalysts, one entity will react or interact with the catalyst to produce a product, while the catalyst remains unchanged.

One way to construct sites 14 and 16 to optimize the time averaged distance between the sites is by using techniques associated with cyclic molecules as described generally in Ernest L. Eliel, Stereochemistry of Organic Compounds, John Wiley & Sons, Inc. pp. 675-685, the complete disclosure of which is herein incorporated by reference. When constructing sites 14 and 16, an appropriate number of bonds may be provided to increase the probability that the chemical entities of libraries A and B will be placed in close enough contact to permit reactions to occur. One particular, non-limiting example of how to product a construct having a pair of sites branching from an arm is illustrated in FIG. 7A. FIG. 7B illustrates one alternative construct having a pair of sites linking members of two different chemical libraries.

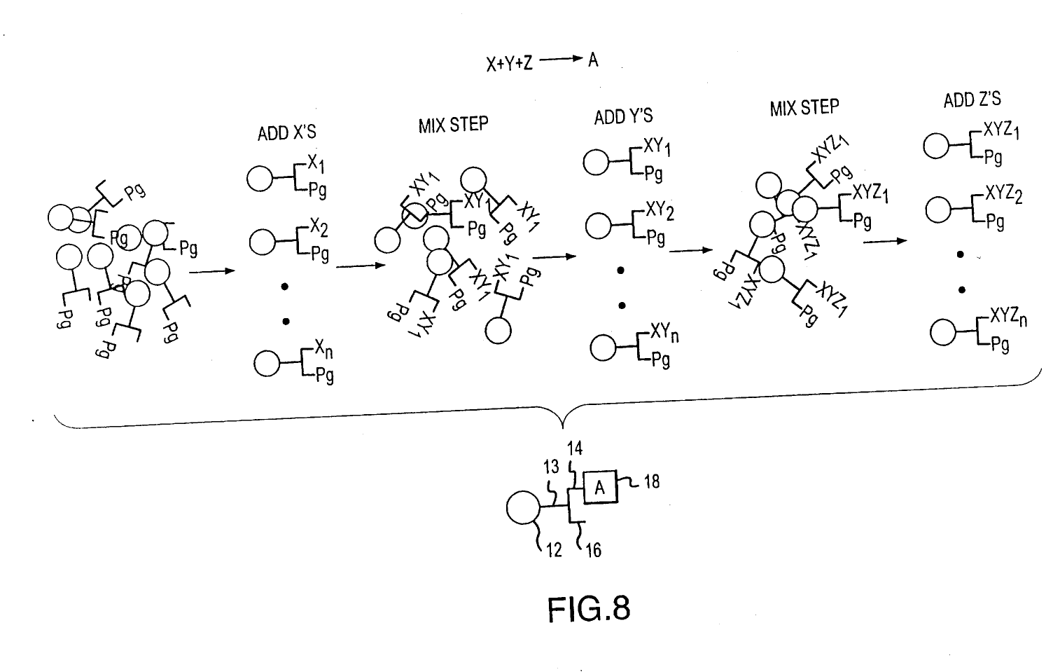

Conveniently, chemical libraries A and B may each be created using a combinatorial process where each chemical entity is formed from two or more chemical building blocks. For example, as shown in FIG. 8, each chemical entity 18 of library A may be formed from three sets of chemical building blocks X, Y and Z. In such as case, chemical building blocks X₁-Xₙ are initially synthesized to site 14. Solid supports 12 are then mixed and split into groups where chemical building blocks Y₁-Yₙ are synthesized to site 14. This process is repeated for building blocks Z₁-Zₙ. Once library A has been synthesized, a similar process may be used for the B library if it is to be constructed from multiple building blocks.

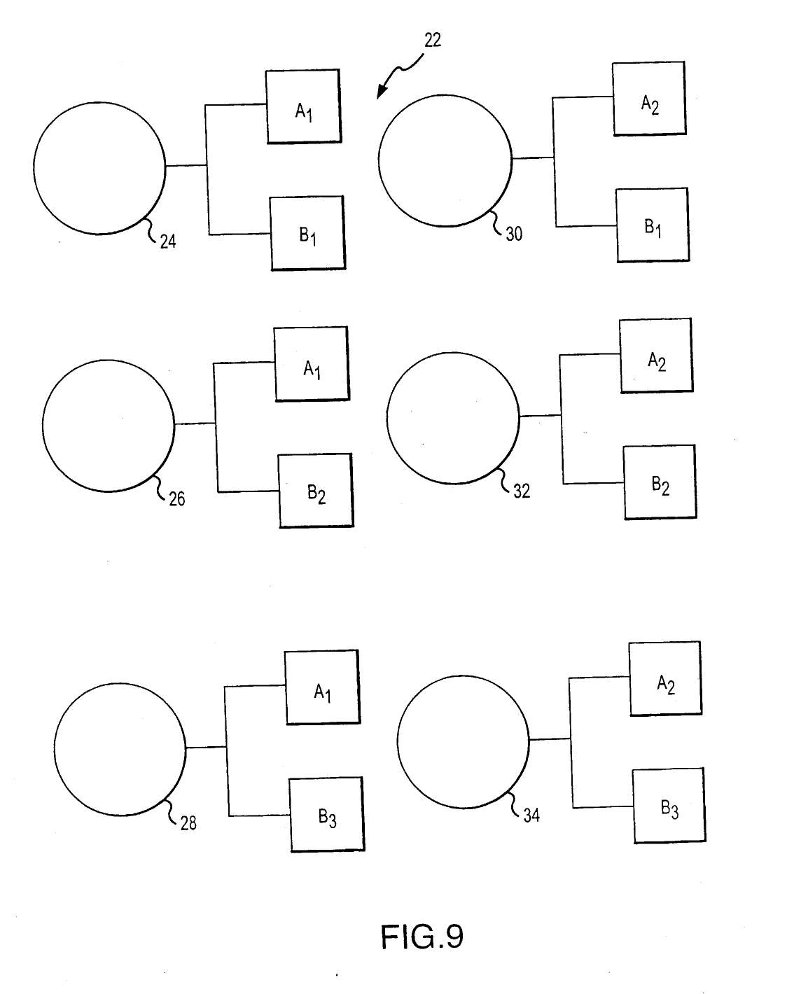

FIG. 9 illustrates one simplified example of a library 22 of constructs 24-34 formed from two chemical libraries A and B, with library A having chemical entities A₁ and A₂, and library B having chemical entities B₁, B₂ and B₃. In so doing, it will be appreciated that, in practice, both libraries may be significantly larger. Library 22 is constructed such that each construct includes a different pairwise combination of chemical entities from libraries A and B. In other words, a given chemical entity from library A will be associated with every one of the chemical entities from library B (on different constructs), and vice versa. As such, the number of constructs is determined by 2×3=6. Hence, library 22 may be constructed so that it is a combination of two or more separate combinatorial libraries.

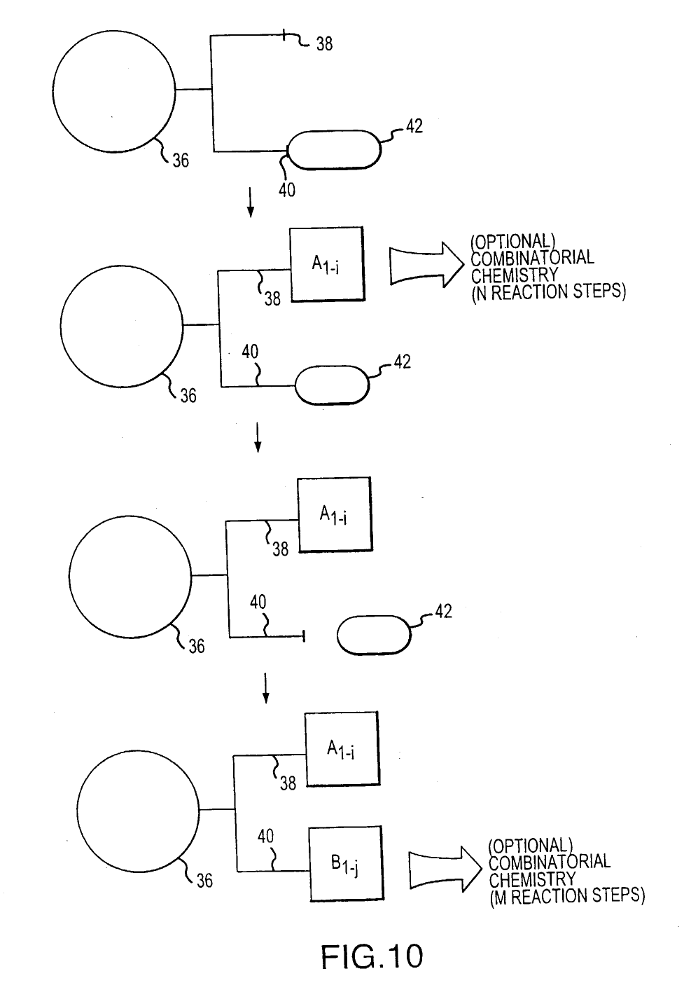

FIG. 10 illustrates one method for forming library 22 of FIG. 9. Initially, a large number of solid supports are provided, with only one solid support 36 being shown for convenience of illustration. Solid support 36 includes a pair of sites 38 and 40. The solid supports are configured such that sites 40 are provided with a protecting group 42. Using a synthesis process, site 38 of each solid support receives a chemical entity A₁₋ᵢ of a library A. Optionally, a combinatorial synthesis process may be employed if the chemical entities contain more than one building block. For example, three mix and split processes may be used so that each chemical entity has three building blocks. Protecting group 42 is then removed from each site 40, and a synthesis process is employed to synthesize chemical entities B₁₋ⱼ of a library B onto sites 40. This may be accomplished, for example, by forming groups of constructs that each include a complete set of members from library A. Each of these groups then receives a different member of library B. If library B is to be constructed of more than one building block, one or more combinatorial processes may be employed in a manner similar to that previously described.

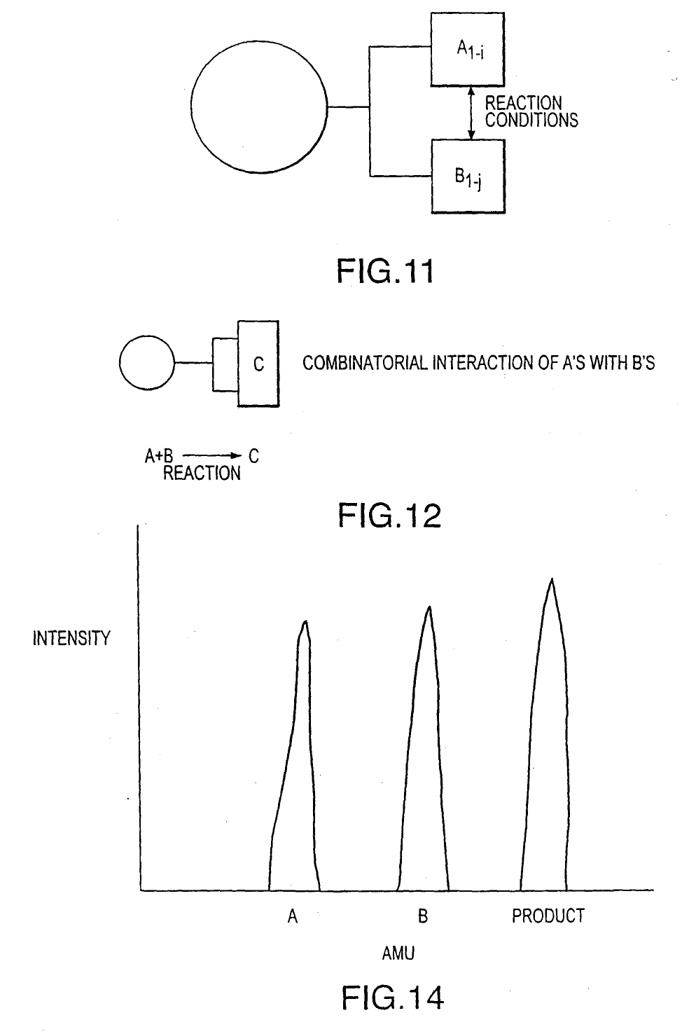

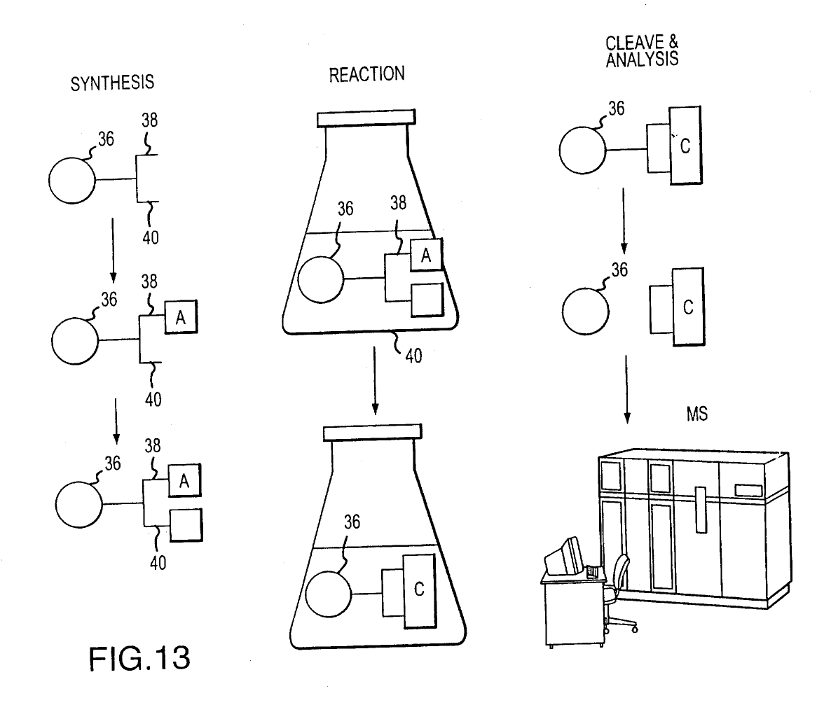

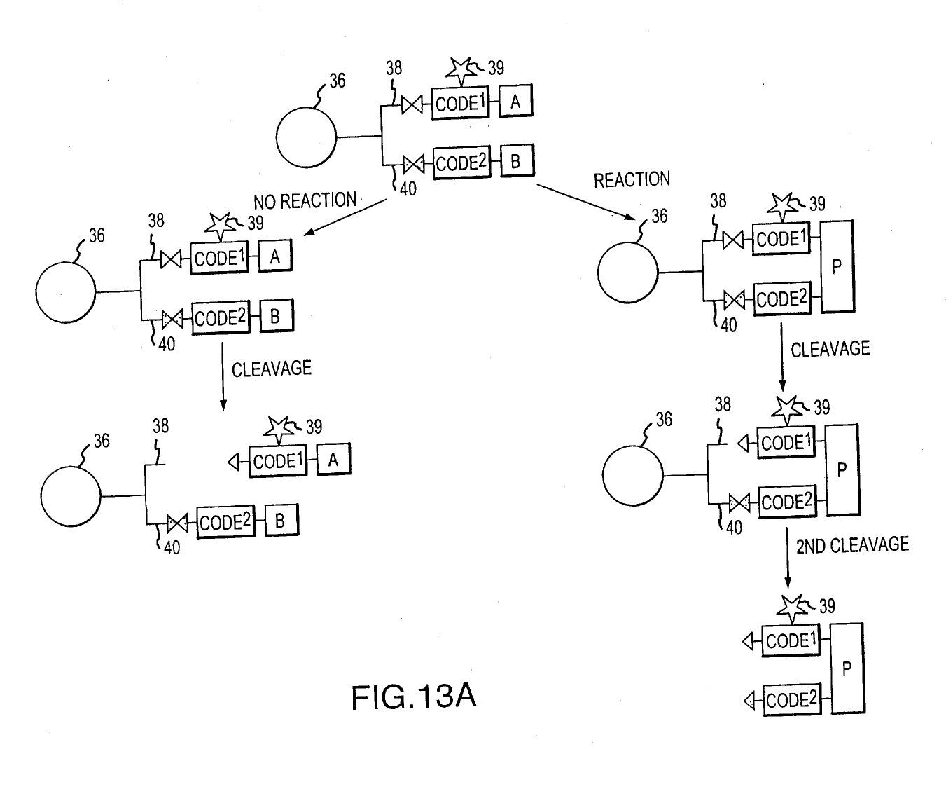

As shown in FIG. 11, once the library of constructs has been created, the constructs are placed under a certain set of conditions to facilitate reactions or interactions. In one application, all of the constructs are placed under the same set of conditions. Such conditions may include, for example, a certain temperature and a certain reagent. Under such conditions, at least some of the constructs will have one of their chemical entities react or interact with the other chemical entity to form a product C (assuming one of the libraries does not contain catalysts) as shown in FIG. 12. A summary of this process involving the construct of FIG. 10 is illustrated in FIG. 13.

To determine which constructs experienced reactions or interactions, a screening process may be performed. For example, one screening process is a thermography process to detect changes in temperature of the constructs to indicate that a reaction has occurred. Other techniques include mass measurement of any chemical products, luminescence or phosphorescence resulting from the creation of a product, colored chromophotography, and the like. The constructs where a reaction or interaction was detected may then be separated from the remainder of the constructs for further evaluation. A variety of separating techniques may be used, including the use of a bead picker.

One specific example of a screening technique is illustrated in FIG. 13A. In FIG. 13A, solid support 36 is shown with sites 38 and 40. A mass identification code is linked between the chemical entities of libraries A and B as described in greater detail with reference to FIG. 16. Also linked to site 38 is a label 39 of some description. If no reaction occurs, cleavage of chemical entity A removes label 39 from site 38 as shown. When construct 36 is scanned, label 39 will not be detected, thus indicating that no reaction or interaction occurred. On the other hand, if a reaction or interaction does occur, cleavage at site 38 will not release label 39 from construct 36 as shown. Hence, when construct 36 is scanned, label 39 will be detected to indicate that a reaction or interaction has occurred. A variety of labels may be used to label the constructs, including, for example, immunological labels, radio isotope labels, chromophore labels, and the like.

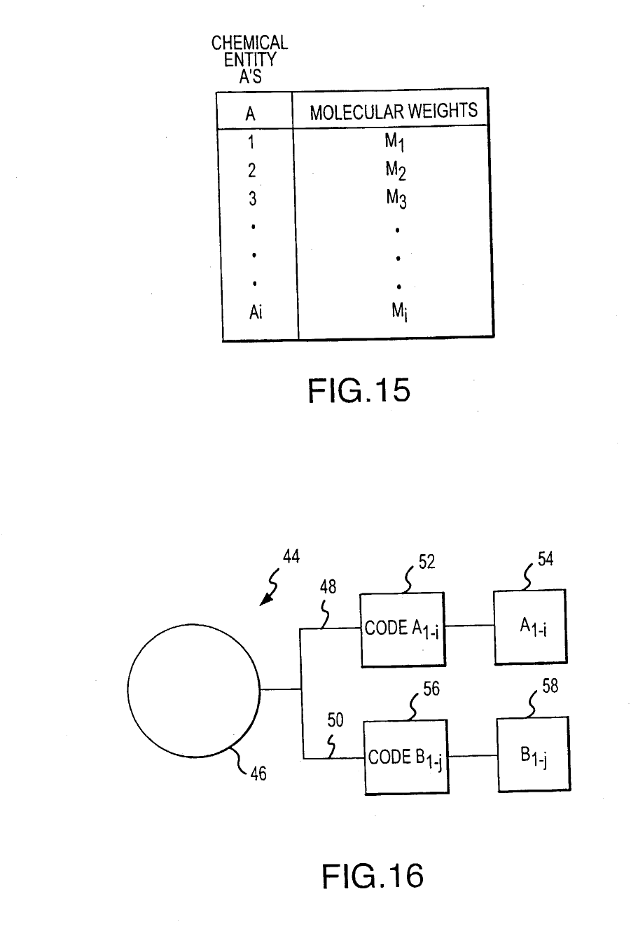

Once the constructs have been separated, the chemical entities of each construct are then identified. As shown in FIG. 13, product C is cleaved from solid support 36 to facilitate evaluation. One convenient way to then identify the chemical entities is by a mass deconvolution process using mass spectroscopy (MS) where it is assumed that at least some of the chemical entities have not reacted and remain attached to the solid support. Further, with such a process, it is assumed that none of the chemical entities is isobaric. The products and remaining chemical entities cleaved from the solid supports are placed in a mass spectrometer where the atomic mass of each chemical entity and product is measured. The mass spectrometer may further be configured to produce a graph illustrating the outcome. One example of such a graph is illustrated in FIG. 14. A look-up table, such as the table of FIG. 15, may then be employed to determine the mass for each chemical entity of the A library. A similar process occurs for the B library.

In this way, two relatively large combinatorial chemical libraries may be reacted with each other, and any reactions or interactions identified in a rapid and efficient manner. Merely by way of example, two combinatorial libraries of 1,000 members each may be reacted with each other to produce a 1,000,000 member library. This library may be rapidly screened for chemical activity, and then, using mass deconvolution, may have the reactive or interactive chemical entities rapidly identified.

An alternative way to identify the chemical entities on the solid supports following screening is by the use of mass codes. FIG. 16 illustrates one example of a construct 44 that includes such mass codes and may be used to create a library formed from multiple combinatorial chemical libraries. Construct 44 comprises a solid support 46 having a pair of sites 48 and 50 similar to the other constructs described herein. Coupled to site 48 is a mass code 52 which in turn is coupled to a chemical entity 54 from a chemical library A. Coupled to site 50 is a mass code 56 which in turn is coupled to a chemical entity 58 from a chemical library B. Each mass code is assigned to a specific chemical entity and is stored in a look-up table as described hereinafter.

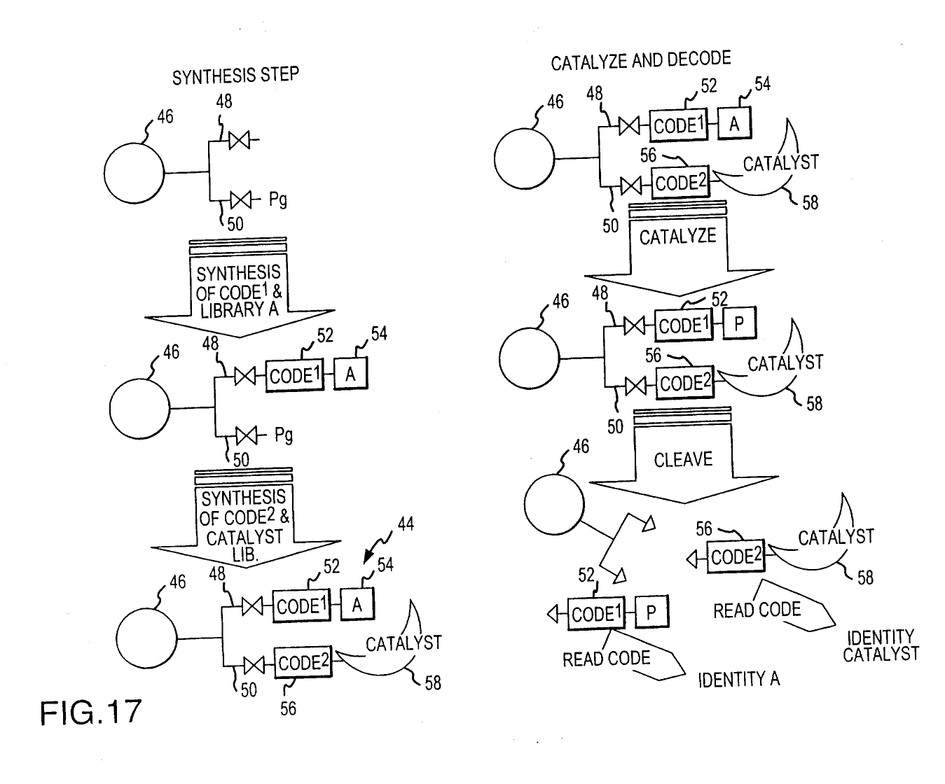

FIG. 17 is a summary of the process used to produce construct 44 (when library B comprises catalysts). To form construct 44, mass code 52 is linked to site 48 and chemical entity A is synthesized to mass code 52 while site 50 is blocked with a protecting group. Site 50 is then unblocked and mass code 56 is linked and chemical entity 58 is synthesized. The manner in which the mass codes may be assigned and linked, as well as techniques for combinatorially synthesizing the chemical entities are described in PCT International Application No.PCT/US97/05701, and in H. Mario Geysen, et al., Isotope or Mass Encoding of Combinatorial Libraries, Chem. & Biol. Vol. III, No. 8, pp. 679-688, August 1996, previously incorporated by reference.

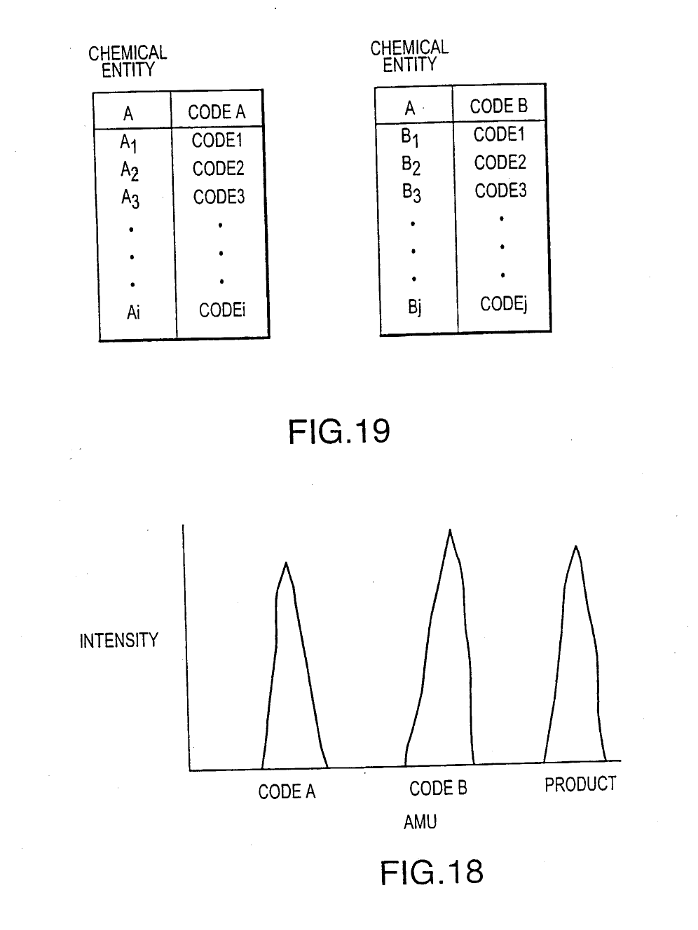

After synthesizing the chemicals onto constructs 44, the constructs are subjected to certain reaction conditions and the constructs are screened for any chemical activity in a manner similar to that previously described. For constructs where chemical activity is found, the codes, catalyst and any products are cleaved and placed into a mass spectrometer to measure the atomic mass of the codes. Conveniently, the mass spectrometer may be configured to graphically display the results as illustrated in FIG. 18. Look-up tables, such as those illustrated in FIG. 19, may then be employed to relate the atomic mass of each measured code to a specific code. In turn, the identified code may be correlated with the chemical entity as described in PCT International Application No. PCT/US97/05701, and in H. Mario Geysen, et al., Isotope or Mass Encoding of Combinatorial Libraries, Chem. & Biol. Vol. III, No. 8, pp. 679-688, August 1996, previously incorporated by reference.

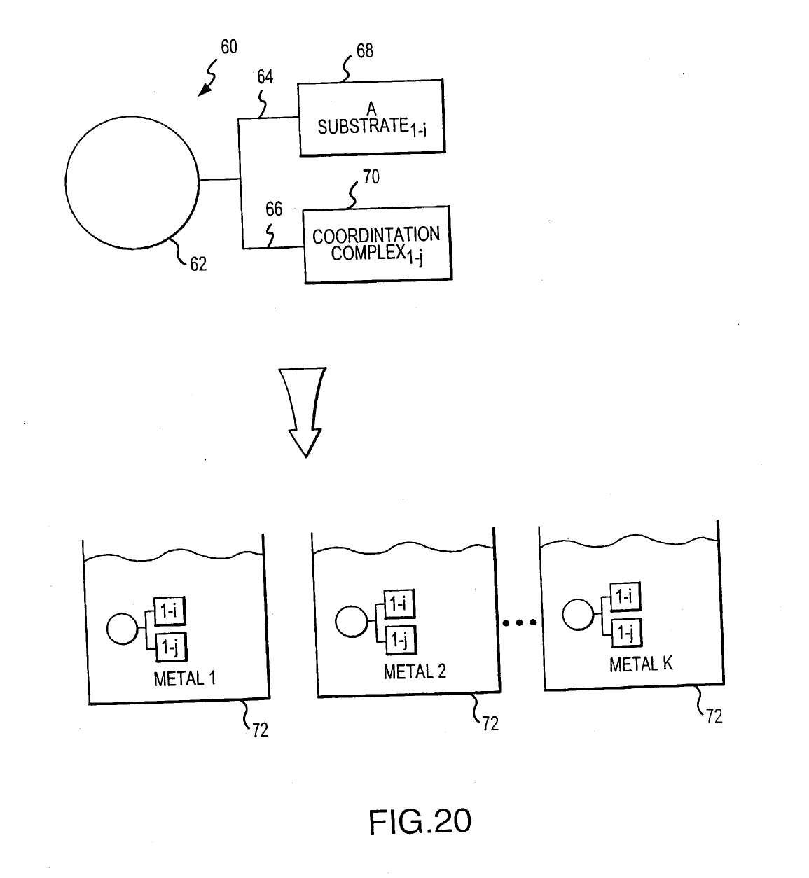

FIG. 20 illustrates an example of a third order combinatorial process. This specific process utilizes a construct 60 that comprises a solid support 62 having a pair of sites 64 and 66. Synthesized to site 64 is a substrate 68 that is part of a combinatorial library of substrates A. Synthesized to site 66 is a coordination complex 70 that is part of a library of coordination complexes. In this way, a library of constructs may be created with every possible pairwise combination of substrates and coordination complexes in a manner similar to that previously described. As shown in FIG. 10, k such libraries are created, with each library placed into a discrete vessel 72. Each vessel then receives a metal that is in one of its oxidation states and that is to be placed into the center of the coordination complexes. Hence, by providing i substrates, j coordination complexes, and k metals, a third order combinatorial library may be created having i×j×k members.

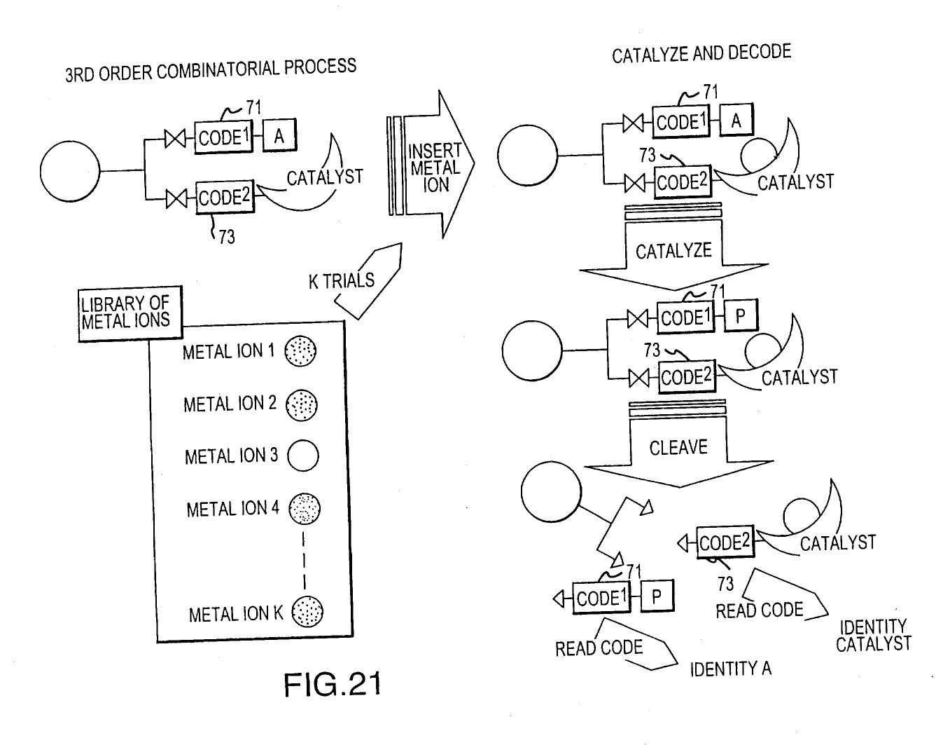

After introducing the metals, the constructs may be screened for chemical activity in a manner similar to that previously described. For constructs where chemical activity occurred, the associated metal may be easily be determined since the last step was performed as a discrete step, i.e., simply identify the vessel where the construct was obtained. The substrate and the coordination complex may be identified using a mass spectrometer and by the use of mass codes in a manner similar to that previously described. FIG. 21 illustrates a similar process with the use of mass codes 71 and 73 to assist in identifying the members of library A and the catalysts in a manner similar to that previously described.

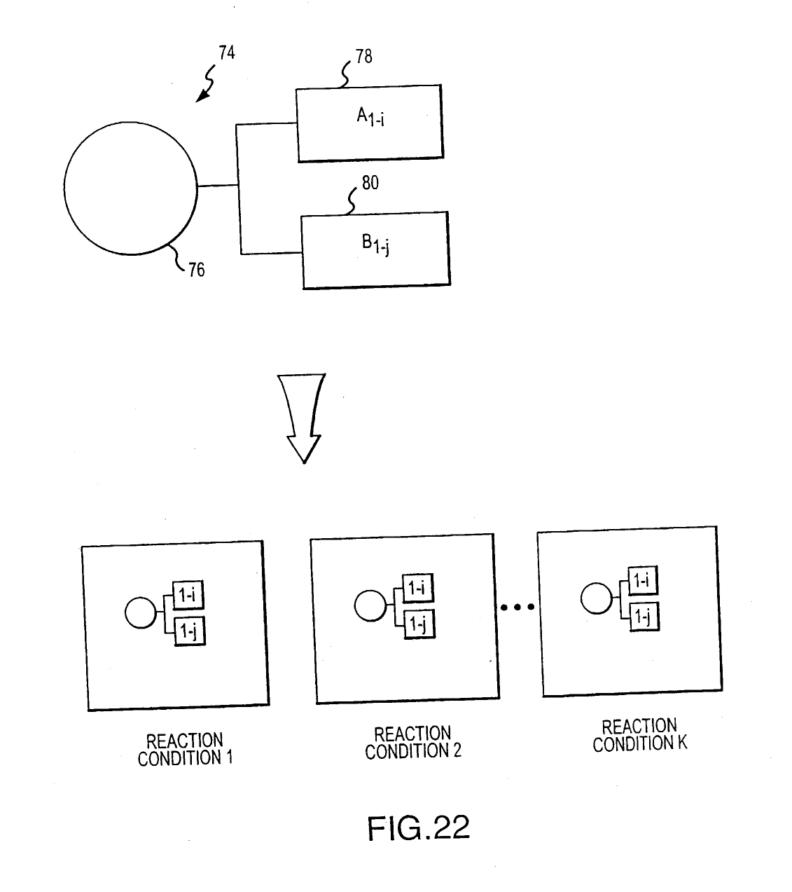

FIG. 22 illustrates another example of a third order combinatorial process. The process of FIG. 22 is similar to that of FIG. 20 in that the last step is performed as a discrete step. The process of FIG. 22 utilizes a construct 74 that comprises a solid support 76 and a chemical entity 78 from a combinatorial library A and a chemical entity 80 from a combinatorial library B. Construct 74 may be constructed in a manner similar to that previously described and may optionally include one or more mass codes in a manner similar to that previously described. Similar to the process of FIG. 20, k libraries of constructs are produced that each include constructs with every possible pairwise combination of chemical entities from libraries A and B. Each comprehensive library is then subjected to a different set of conditions as a discrete step as shown. In this way, i chemical entities from a library A may be reacted with j chemical entities from a library B, each under k conditions. The constructs are then screened and chemical entities of interest (and associated reaction conditions) identified in a manner similar to that previously described.

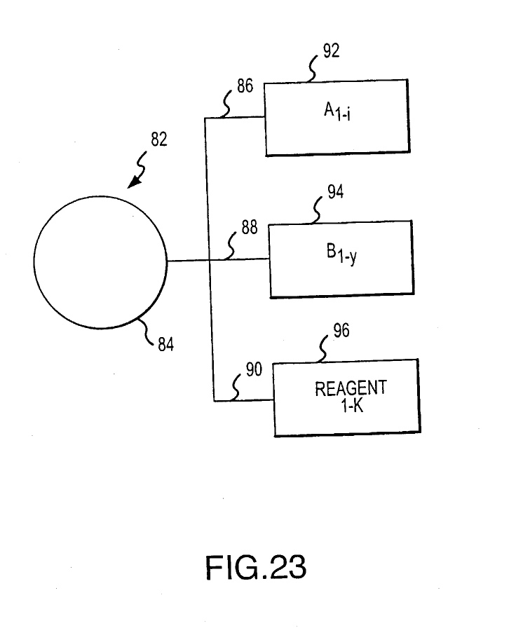

The invention further provides constructs that include more than two sites. In this way, n^{th} order combinatorial processes may be performed. An example of such a construct 82 is illustrated in FIG. 23. Construct 82 comprises a solid support 84 having three sites 86, 88 and 90. A chemical entity 92 from a library A (which may optionally be produced combinatorially from multiple building blocks) is linked to site 86, and a chemical entity 94 from a library B (which may optionally be produced combinatorially from multiple building blocks) is linked to site 88 in a manner similar to that described with previously embodiments. A reagent 96 from a library of reagents is linked to site 90.

With the use of constructs 82, i chemical entities from a library A may be reacted with j chemical entities from a library B using k reagents. Screening and deconvolution, including the use of mass codes, may be performed in a manner similar to that previously described.

In another aspect of the invention, constructs may be formed that have catalysts from two or more catalyst libraries to determine which combinations of catalysts are the most efficient in producing end products. For example, a set of constructs may be configured such that each construct of the set includes a pairwise combination of a chemical entity A₁-Aᵢ of a catalyst library A and a chemical entity B₁-Bⱼ of a catalyst library B. Conveniently, the set of constructs may include essentially every possible pairwise combination of the chemical entities A₁-Aᵢ of the catalyst library A and the chemical entities B₁-Bⱼ of the catalyst library B.

The constructs are exposed to a substrate in solution phase to facilitate potential reactions or interactions involving the chemical entity A₁-Aᵢ and the chemical entity B₁-Bⱼ of each construct. The constructs may then be screened to identify any reactions or interactions, and the chemical entity A₁-Aᵢ and the chemical entity B₁-Bⱼ of any constructs where reactions or interactions occurred may be identified. In this way, a comprehensive analysis of the interaction of two catalyst libraries with a substrate may be performed.

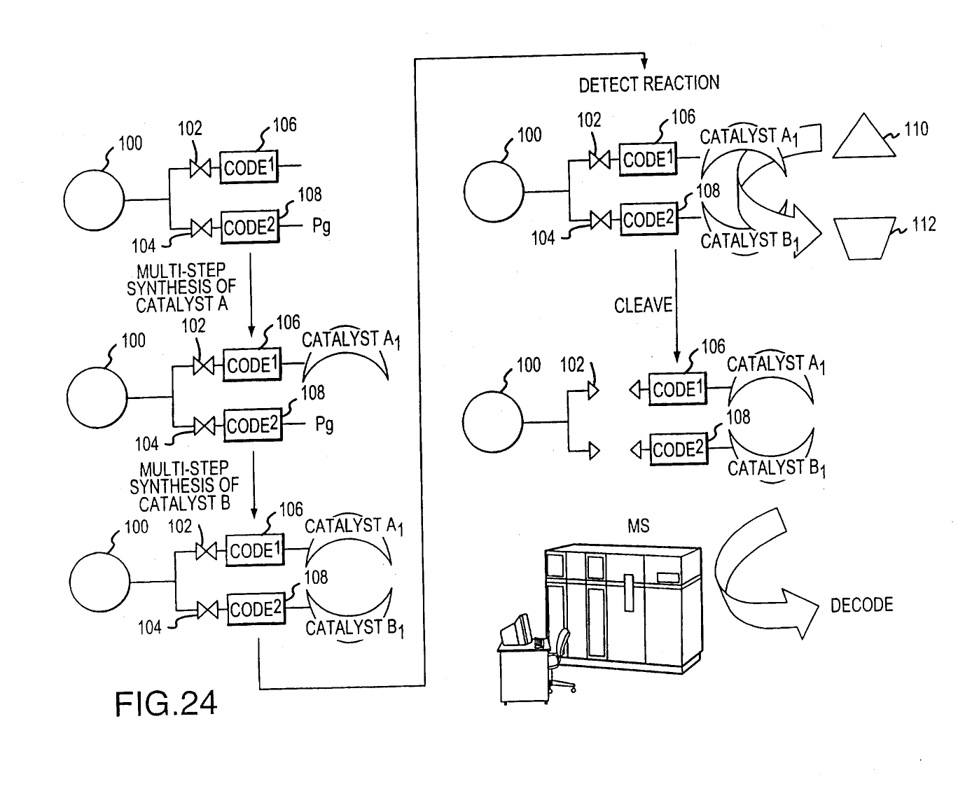

One example of such a process is illustrated in FIG. 24. The process employs a plurality of solid supports 100 (only one being illustrated for convenience of discussion). Solid support 100 has a pair of tether sites 102 and 104 that may be constructed in a manner similar to the other embodiments described herein. Optionally, mass codes 106 and 108 may be linked to each site 102 and 104, respectively, in a manner similar to other embodiments and used to identify a particular catalyst after a reaction has been identified. Initially, site 104 includes a protecting group (Pg) and a member A₁ of a catalyst library A is synthesized to site 102. As with other embodiments described herein, a multi-step synthesis process may be used to produce member AI. Although not shown, each solid support may receive a different member of the catalyst library A in a manner similar to other embodiments.

Protecting group Pg is then removed and the above process is repeated to synthesize a member B I of a catalyst library B to site 104. In this way, a set of constructs may be produced with every pairwise combination of catalysts from libraries A and B. The set of constructs is then exposed to a substrate 1 10 to potentially produce a product 112 if a reaction occurs. The set of constructs may then be screened for potential reactions or interactions using any of the screening techniques described herein. For constructs where reactions or interactions are detected, codes 106 and 108 and catalyst members A₁ and B₁ may be cleaved from solid support 100. Codes 106 and 108 may then be decoded using mass spectroscopy in a manner similar to that previously described to identify the particular catalysts involved in the reaction.

The invention has now been described in detail for purposes of clarity and understanding. However, it will be appreciated that certain changes and modifications may be practiced within the scope of the appended claims.

What is claimed is:

1. A method to screen for reactive chemicals, the method comprising:
configuring a set of constructs such that each construct of the set includes a pairwise combination of a chemical entity A₁-Aᵢ of a chemical library A and a chemical entity B₁-Bⱼ of a chemical library B, with the set of constructs including essentially every possible pairwise combination of the chemical entities A₁-Aᵢ of the chemical library A and the chemical entities B₁-Bⱼ of the chemical library B;
exposing the constructs to a given set of conditions to facilitate reactions or interactions between the chemical entity A₁-Aᵢ and the chemical entity B₁-Bⱼ of each construct;
screening the constructs to identify any reactions or interactions; and
determining the chemical entity A₁-Aᵢ and the chemical entity B₁-Bⱼ of any constructs where reactions or interactions occurred.

2. A method as in claim 1, wherein each construct includes at least a pair of sites, and further comprising synthesizing the chemical entity A₁-Aᵢ to one of the sites of each construct while the other site is blocked, unblocking the other site of each construct, and then synthesizing the chemical entity B₁-Bⱼ to the other site of each construct.

3. A method as in claim 2, further comprising mixing the constructs after synthesizing the chemical entities A₁-Aᵢ, splitting the constructs into groups such that each group has constructs with essentially all other chemical entities A₁-Aᵢ, and synthesizing the chemical entities B₁-Bⱼ onto the constructs such that each group receives a different chemical entity B₁-Bⱼ.

4. A method as in claim 2, wherein the synthesizing steps comprise synthesizing a combination of chemicals onto each construct to create each A₁-Aᵢ chemical entity and/or each B₁-Bⱼ chemical entity.

5. A method as in claim 4, further comprising mixing the constructs and splitting the constructs into groups as each chemical of the combination is synthesized.

6. A method as in claim 1, wherein the screening step comprises sensing for a change in temperature to indicate that a reaction or an interaction has occurred or mass measuring for any chemical products.

7. A method as in claim 1, wherein the determining step comprises evaluating the masses of the unreacted chemical entities A₁-Aᵢ and the unreacted chemical entities B₁-Bⱼ using mass spectrometry and correlating each mass with an associated chemical entity of each library.

8. A method as in claim 1, further comprising encoding each chemical entity A₁-Aᵢ and each chemical entity B₁-Bⱼ with a code, and wherein the determining step comprises decoding the codes.

9. A method as in claim 8, wherein the decoding step comprises evaluating the mass of the codes using mass spectrometry and correlating each mass with an associated chemical entity.

10. A method as in claim 1, wherein chemical library A or chemical library B comprises catalysts.

11. A method as in claim 10, further comprising providing multiple libraries of constructs that each include the same pairwise combinations of chemical entities A₁-Aᵢ chemical entities B₁-Bⱼ, and further comprising exposing each library of constructs to a metal in one of its oxidation states.

12. A method as in claim 1, further comprising providing multiple libraries of constructs that each include the same pairwise combinations of chemical entities A₁-Aᵢ chemical entities B₁-Bⱼ, and further comprising exposing each library of constructs to a different set of conditions.

13. A method for making a library of constructs, the method comprising:
providing a set of solid supports that each include at least two sites;
synthesizing a chemical entity from a chemical library A having A₁-Aᵢ chemical entities to one of the sites of each solid support while the other site is blocked;
unblocking the blocked site for each solid support; and
synthesizing a chemical entity from a chemical library B having B₁-Bⱼ chemical entities to the unblocked sites to form a set of constructs that includes essentially every possible pairwise combination of the chemical entities A₁-Aᵢ of the chemical library A and the chemical entities B₁-Bⱼ of the chemical library B.

14. A method as in claim 13, further comprising mixing the constructs after synthesizing the chemical entities A₁-Aᵢ, splitting the constructs into groups such that each group has constructs with essentially all other chemical entities A₁-Aᵢ, and synthesizing the chemical entities B₁-Bⱼ onto the constructs such that each group receives a different chemical entity B₁-Bⱼ.

15. A method as in claim 13, wherein the synthesizing steps comprise synthesizing a combination of chemicals on each construct to create each A₁-Aᵢ chemical entity and/or each B₁-Bⱼ chemical entity.

16. A method as in claim 15, further comprising mixing the constructs and splitting the constructs into groups as each chemical of the combination is synthesized.

17. A method as in claim 13, further comprising encoding each chemical entity A₁-Aᵢ and each chemical entity B₁-Bⱼ with an identification code.

18. A construct comprising:
a solid support having at least one arm and at least a pair of sites branching from the arm;
a chemical entity A coupled to one of the sites; and
a chemical entity B coupled to the other site, with the pair of sites being configured such that the chemical entity A is spaced apart from the chemical entity B at a distance selected to facilitate a reaction between the chemical entity A and the chemical entity B.

19. A construct as in claim 18, further comprising an identification code coupled to the chemical entity A and the chemical entity B.

20. A chemical construct library, comprising:
a set of constructs that each comprise a solid support having at least one arm and at least a pair of sites branching from the arm, a chemical entity A₁-Aᵢ of a chemical library A coupled to one of the sites, and a chemical entity B₁-Bⱼ of a chemical library B coupled to the other site, with the pair of sites being configured such that each chemical entity A₁-Aᵢ is spaced apart from each chemical entity B₁-Bⱼ at a distance selected to facilitate a reaction or an interaction between each chemical entity A₁-Aᵢ and each chemical entity B₁-Bⱼ.

21. A library as in claim 20, wherein the set of constructs includes essentially every possible pairwise combination of the chemical entities A₁-Aᵢ of the chemical library A and the chemical entities B₁-Bⱼ of the chemical library B.

22. A library as in claim 21, wherein the chemical entities A₁-Aᵢ of the chemical library A and/or the chemical entities B₁-Bⱼ of the chemical library B each comprise multiple chemical building blocks that have been synthesized to the sites.

23. A library as in claim 20, wherein the library A or the library B comprises catalysts.

24. A chemical construct library comprising:
a set of constructs that each comprise a solid support having at least a pair of sites, a chemical entity A₁-Aᵢ of a chemical library A coupled to one of the sites, wherein the chemical entity A₁-Aᵢ comprises two or more chemical building blocks that have been synthesized to the site, and a chemical entity B₁-Bⱼ of a chemical library B coupled to the other site.

25. A library as in claim 24, wherein the set of constructs includes essentially every possible pairwise combination of the chemical entities A₁-Aᵢ of the chemical library A and the chemical entities B₁-Bⱼ of the chemical library B.

26. A library as in claim 24, wherein the library A or the library B comprises catalysts.

27. A method to screen for reactive chemicals, the method comprising:
configuring a set of constructs such that each construct of the set includes a pairwise combination of a chemical entity A₁-Aᵢ of a catalyst library A and a chemical entity B₁-Bⱼ of a catalyst library B;
exposing the constructs to a substrate in solution phase to facilitate potential reactions involving the chemical entity A₁-Aᵢ and the chemical entity B₁-Bⱼ of each construct;
screening the constructs to identify any reactions or interactions; and
determining the chemical entity A₁-Aᵢ and the chemical entity B₁-Bⱼ of any constructs where reactions or interactions occurred.

28. A method as in claim 27, wherein the set of constructs include essentially every possible pairwise combination of the chemical entities A₁-Aᵢ of the catalyst library A and the chemical entities B₁-Bⱼ of the catalyst library B
